(19) United States
(12) Patent Application Publication
(10) Pub. No.: US 2011/0106186 A1
(43) Pub. Date: May 5, 2011

(54) BONE FRAGMENT EXTRACTION

(76) Inventor: Nikolaj Wolfson, San Francisco, CA (US)

(21) Appl. No.: 12/611,071

(22) Filed: Nov. 2, 2009

(51) Int. Cl. A61B 17/56

(52) U.S. Cl. 606/86 R

(57) ABSTRACT

Methods and structures for removing a bone fragment from a body of a patent. A method can include advancing a distal portion of an orthopedic extraction device into a bone fragment disposed in a bone mass in the body of the patient, and actuating one or more deployable members of the distal portion of the device positioned in the bone fragment so as to engage the bone fragment with the device. With engagement between the device and bone fragment, a force or torque to the device so as to remove the bone fragment from the bone mass of the patient.

### BACKGROUND OF THE INVENTION

The present invention relates generally to bone fragment removal structures and methods. More particularly, in one application the present invention provides methods and structures for removal of a head of a femur from the acetabulofemoral joint of a patient.

The hip joint, or acetabulofemoral joint, is an articulation between the head of the femur and the cup-like acetabulum. The joint is generally referred to as a spheroid or ball and a socket joint, with the average radius being about 25 mm, and generally ranging from about 20 mm to about 36 mm in radius (Theme Atlas of Anatomy, 2006). The head of the femur is largely contained within the acetabulum, and is held within it by the soft tissue structures inside the acetabulum (ligamentum teres) and outside of it, at its rim, by the cartilaginous ring-shaped structure is referred to as the acetabulum labrum. The femoral head is also held in place by the capsule of the hip joint, which is attached to the acetabulum and to the area just below the head of the femur, so called “neck of the femur”.

In some circumstances, permanent removal of the head of the femur is surgically accomplished. In one instance, removal of the femoral head is accomplished when the neck of the femur is fractured and hip replacement is required. Such femoral fracture and corresponding hip replacement surgery is typically, though not exclusively, encountered in the elderly population. Femoral head removal is also accomplished as part of the hip replacement surgery in the absence of specific fracture, but where substantial hip degeneration has occurred. With the anterior hip replacement approach in the absence of specific fracture, osteotomy of the neck of the femur is performed first to facilitate removal of the head of the femur so hip replacement can be performed. In some instances, dislocation of the femoral head from the socket is accomplished during a hip replacement procedure absent fracture or prior to osteotomy.

In hip replacement procedures, whether including a femoral head fracture or a neck osteotomy, extraction of the femoral head from the acetabulum is necessary to perform the replacement procedure. Extraction of the head of the femur from the acetabulum, however, is often a challenging procedure due to a variety of factors, including the anatomy of the joint and attachment of the head of the femur to the acetabulum holding the components of the joint securely in place. Regarding the joint attachment, the most inner part of the head and the acetabulum are connected to each other via ligamentum teres, while the cartilaginous rim of the acetabulum, known as labrum, and the capsule of the hip joint hold the head of the femur within the acetabulum.

The traditional way of removal of the head of the femur from the acetabulum is based on the use of the so-called corkscrew device. The orthopedic corkscrew is a conical or spiral tool, having a treaded end and which is inserted into the head of the femur by bringing the distal tip into forced contact with the femoral head and applying rotational movement so as to screw the device into the femoral head. After the corkscrew is introduced deep into the head of the femur, the device is pulled outside the acetabulum together with the femoral head.

Both insertion of this type of device into femoral head and extraction of the head of the femur from the acetabulum are often either limited in effectiveness or fail due to number of factors. First, the traditional corkscrew type devices are based on a rotational mechanism of insertion. Because a hip joint, by anatomical design, allows the femoral head to move within the acetabulum, application of a rotational force in use of such a device tends to elicit spinning or rotational movement to the head of the femur, making advancement of the device into the bone difficult and cumbersome. Use of additional tools, e.g., gripping or positioning devices, are often necessary to counter the undesired rotation or movement of the femoral head, further complicating the procedure. Additionally, corkscrew device purchase in the bony structure is often not sufficiently strong, e.g., due to the variable quality of the bone density. This is particularly problematic in patients with lower bone density patients, and commonly encountered in elderly, malnourished, and other categories of patients. As bone density in a patient decreases, the pull out strength, and therefore, effectiveness, of the existing types of removal device construct diminishes. In such instances, attempt to remove the head of the femur with application extraction force to an engaged device can cause failure of the screw/bone interface and the device is removed while the head of the femur, in some cases fragmented, remains within the acetabulum. Repeated attempts to reinsert the corkscrew type of device cause distraction of the bone material leading often to the distraction of the head of the femur on sometimes even to the damage of the acetabulum. Thus, existing methods can be limited in effectiveness and may cause damage to the cartilage of the acetabulum, which is important to preserve, particularly when the goal of surgery is in replacing only the head of the femer, e.g., so-called hemiarthroplasty.

Thus, notwithstanding the variety of efforts in the prior art, there remains a continuing need for orthopedic bone extraction or removal devices with improved engagement and removal force to a bone fragment needing extraction, such as a femoral head disposed in the acetabulum of a patient.

### BRIEF SUMMARY OF THE INVENTION

The present invention includes method and structures for removing a bone fragment from a body of a patent. In one aspect, a method includes advancing a distal portion of an orthopedic extraction device into a bone fragment disposed in a bone mass in the body of the patient, and actuating one or more deployable members of the distal portion of the device positioned in the bone fragment so as to engage the bone fragment with the device. With engagement between the device and bone fragment, a force or torque to the device so as to remove the bone fragment from the bone mass of the patient.

In one application, the method includes bone removal from the acetabulofemoral joint of a patient. In such an embodiment, the bone fragment includes the head of a femur and the bone mass includes the acetabulum. Removal may be selected in the context of a femoral fracture or neck osteotomy, or dislocation of the femoral head from the socket, e.g., performed absent/prior to fracture or osteotomy. Removal according to the present methods may be performed in conjunction with any variety of surgical approaches including, e.g., an anterior, posterior, etc. hip approach; minimally invasive surgical procedure, or any other commonly used approaches. The femoral fracture may be a femoral neck fracture, such as subcapital, midcervical, basicervical fracture and the like. The advancing step can include advancing the distal portion of the device along an axis that extends through an entry point on the surface of the bone and in the direction of the head of the femur. For example, the entry point may be through the neck of the femur (e.g., at a femoral fracture surface) and into the femoral head. A method may optionally include a drilling step, including drilling a bore along the desired axis extending into and/or in the direction of the femoral head. Drilling can be accomplished with use of a drill guide. The distal portion of the device is then advanced along the drilled passageway and into the femoral head, for expansion or deployment of the members to engage the femoral head. Application of a selected force or torque (e.g., pulling, twisting, and/or rotational movement) can cause movement of the head of the femur within the acetabulum so as to free the femur from soft tissue attachments, and pulling or removal of the femoral head from the acetabulum.

In another aspect, the present invention includes orthopedic extraction structures and devices for removing a bone fragment from a bone mass. Various designs and/or configurations of a removal device will be available, according to the present invention. In one example, a device includes a proximal handle configured for user manipulation or position control of the device. Deployable members can be shaped or selected such that one or more members includes a hooked distal portion to facilitate engagement between deployable members and a bone tissue. Deployable members will typically be coupled about a hinge to allow movement relative to other members of the assembly. In one example, deployable members are coupled proximally about a pin hinge, with the assembly couplable to a distal portion of the elongate body about the pin hinge. The deployable member assembly may provide a distal most portion of the device or, alternatively, a threaded or sharpened portion disposed distally to the deployable member assembly. The deployable member assembly may optionally be removably coupled to the elongate body, and the device may include one or more single or limited use components.

The actuation member, as noted above, will be coupled to the deployable member assembly such that movement of the actuation member, or application of a selected force/torque to the actuation member, elicits movement of the deployable member assembly. Various coupling means may be utilized. In one embodiment, the device includes a push rod disposed in a lumen of an elongate body housing. The actuation member can be coupled to the push rod such that manipulation of the actuation member brings a distal portion of the push rod into further contact with the deployable member assembly so as to elicit movement of the assembly.

In yet another aspect, the present invention includes a system for removing a bone fragment from a bone mass. The system can include an orthopedic extraction device and one or more components for drilling or forming a passageway in a bone fragment targeted for removal. For example, a system can include a guide member for directing a drilling or boring device into contact with the bone fragment. In one example, a guide member includes an elongate housing having a proximal portion coupled to a handle and a distal portion configured for engagement with a surface of a bone fragment. In another exemplary embodiment, the guide includes a housing that is a continuous tubular housing or a discontinuous housing comprising substantially opposing prongs.

Structures and methods can include one or more components that are removable coupled or designed for limited or single use applications. For example, a device can include a deployable member assembly that is removable coupled to the elongate body of the device, and the system can include one or more replacement components for the deployable member assembly.

For a fuller understanding of the nature and advantages of the present invention, reference should be made to the ensuing detailed description and accompanying drawings. Other aspects, objects and advantages of the invention will be apparent from the drawings and detailed description that follows.

### BRIEF DESCRIPTION OF THE DRAWINGS

FIGS. 1A through 1C illustrate removal of a femoral head fragment from a acetabulum with a bone fragment removal device, according to an embodiment of the present invention.

FIGS. 2A and 2B illustrate a bone fragment removal device, according to the present invention. FIG. 2A illustrates the device with a distal assembly in a substantially non-deployed state. FIG. 2B shows the device with the distal assembly in a substantially deployed state.

FIG. 3A is a cross-sectional diagram of a bone fragment removal device according to an exemplary embodiment of the present invention.

FIG. 3B shows a simplified diagram of an exemplary deployable member assembly of one embodiment of the present invention.

FIG. 4A is an exploded view of a bone fragment removal device assembly according to an embodiment of the present invention.

FIG. 4B shows a bone fragment removal device, as in FIG. 4A, in an assembled configuration.

FIG. 4C is an enlarged view of portion 4C of FIG. 4B.

FIG. 5A illustrates a drill guide or member for guiding a drilling or boring device into contact with a surface of a bone.

FIG. 5B illustrates a drill guide having a handle and a body including opposing prongs, according to an embodiment of the present invention.

FIG. 5C illustrates a guide directing a drilling device into contact with a fractured femoral head disposed in a acetabulum.

FIG. 6A shows a distal portion of a removal device having a threaded end-section.

FIG. 6B shows a distal portion of a removal device having a sharpened or pointed end-section.

### DETAILED DESCRIPTION OF THE INVENTION

The present invention provides bone fragment removal structures and methods applicable in a wide variety of bones and fractures. The present invention methods and structures are particularly well suited for removal of a bone fragment from the acetabulofemoral joint of a patient, including removal of a femoral head from the acetabulum of the patient, e.g., in a hip replacement procedure.

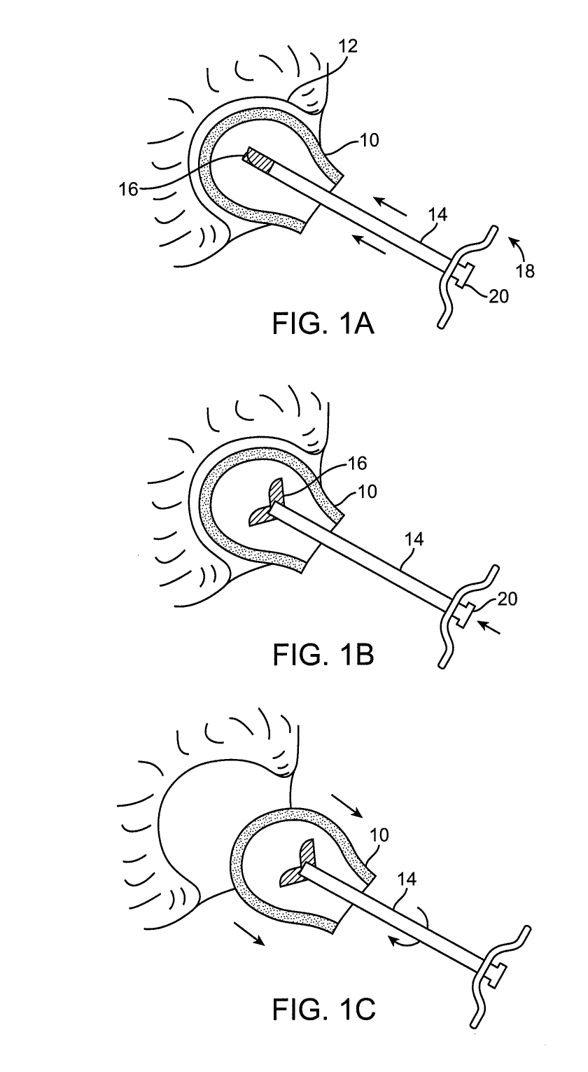

Removal of a bone fragment from a bone mass according to an embodiment of the present invention is illustrated with reference to FIGS. 1A through 1C. FIG. 1A shows a fractured femoral head 10 disposed in the hip joint socket or acetabulum 12 of a patient. An extraction device 14 is provided having an elongate body and a distal portion with an expandable or deployable member assembly 16. The proximal portion 18 of the device includes a handle for controlling or manipulating positioning of the device. The proximal portion further includes an actuation member 20 that is coupled to the deployable member assembly so as to allow control of movement or deployment of elements of the deployable member assembly at the distal portion.

Removal of the bone fragment includes advancing the distal portion of the extraction device 14 into the femoral head fragment 10, for example, as shown in FIG. 1A. The distal portion may be advanced through the fragment tissue or inserted or advanced along a passageway created in the fragment. For example, an orthopedic drilling or boring device may be utilized to form a hole or passage into the bone fragment structure, and the distal portion of the device subsequently inserted into and advanced along the passageway. Device entry can occur at various entry points or locations on the femoral head fragment, and will not be limited to any particular entry location. Following positioning of the device distal portion in the femoral head, the deployable member assembly 16 is actuated so as to engage the bone fragment 10 (e.g., FIG. 1B). Actuation of the assembly 16 can include manipulation of the actuation member 20, for example, via application of a force (e.g., applied distally and/or axially) or torque to the actuation member. As the actuation member 20 will be mechanically coupled to the deployable member assembly 16, manipulation by the user elicits movement or deployment of members of the assembly including, for example, movement of the assembly radially or expanding outward, e.g., from a substantially non-deployed configuration and to a more deployed arrangement that allows the deploying members to push further into contact with the bone tissue and more securely engage the fragment.

With the device 14 engaged with the femoral head fragment 10, movement of the bone 10 can be accomplished by application of force or torque to the device 14, as illustrated in FIG. 1C. For example, a user gripping the handle may apply a twisting and/or pulling movement to the femoral head. Application of force or torque (e.g., twisting or rotational movement) can cause rotation of the head of the femur within the acetabulum so as to free the femur from the soft tissue attachments. With the femoral head freed from the soft tissue attachments, the fragment can be pulled out or removed from the acetabulum with the device.

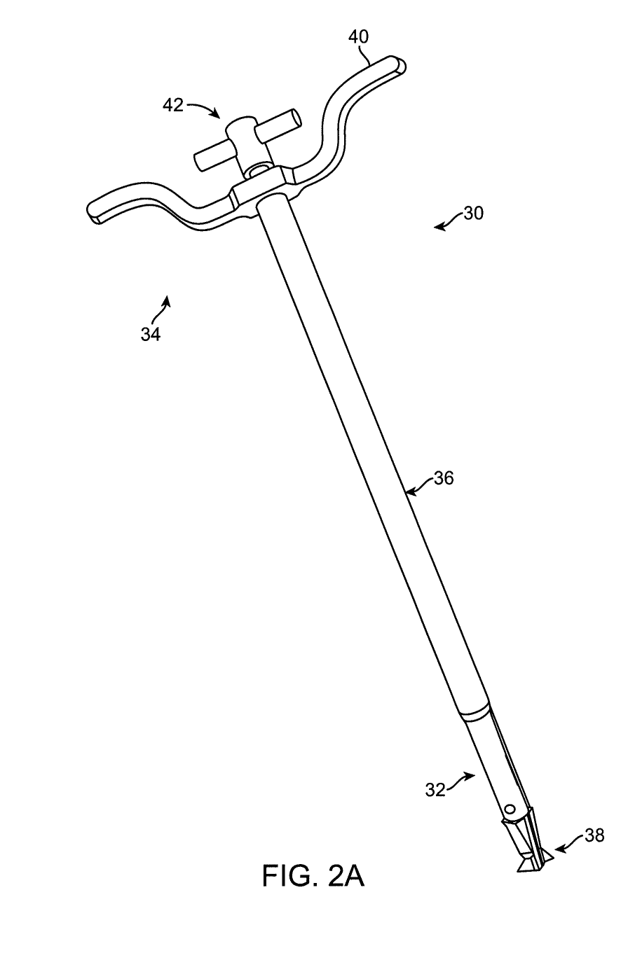

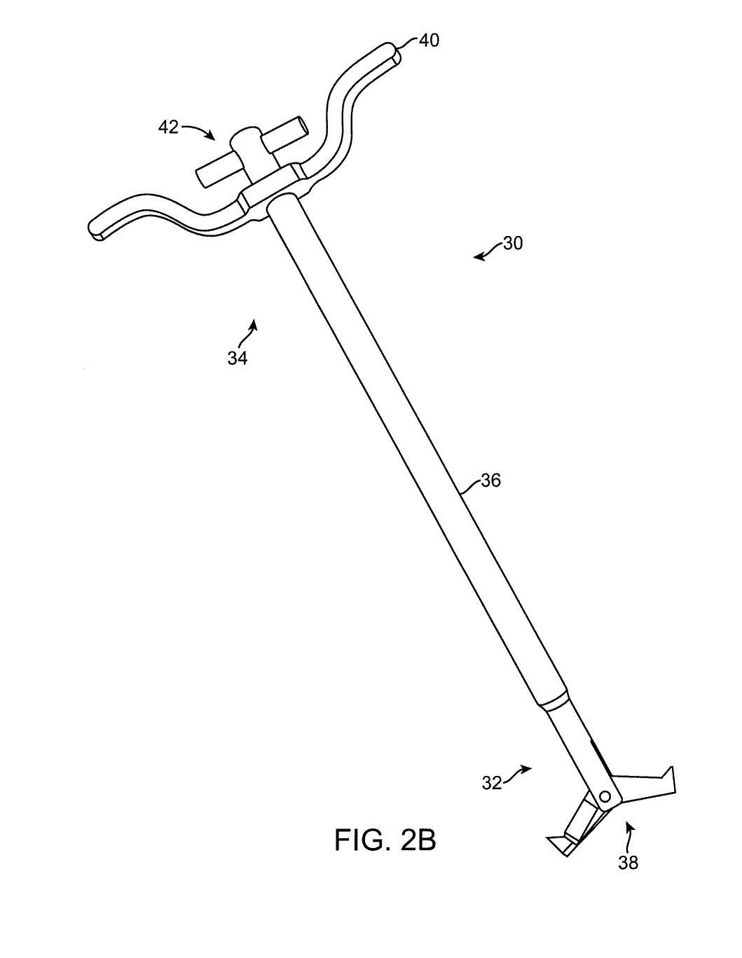

FIGS. 2A and 2B illustrate a bone fragment removal device, according to one exemplary embodiment of the present invention. The device 30 includes a distal portion 32 and a proximal portion 34, with an elongate body 36 coupling the proximal 34 and distal portions 32. The distal portion includes a deployable member assembly 38, the assembly including movable members coupled to the distal end of the elongate body about a pinned hinge. The proximal portion 34 includes a handle 40 for positioning or manipulation of the device and an actuation member 42. The actuation member 42 is mechanically coupled to the deployable member assembly 38 of the distal portion 32, and configured to transmit a force or torque to the deployable member assembly 38 for actuation or movement of the assembly 38.

FIG. 2A illustrates the device 30 with a distal assembly 38 in a substantially non-deployed state. FIG. 2B shows the device 30 with the distal assembly 38 in a substantially deployed state. The members are movable between, and in some instances beyond, the configurations specifically illustrated. In embodiments optimized for use in hip replacement/repair procedures in an adult population, the device and components thereof will be of a construction and design, as well as sizing, suitable for removal of a femoral head from an acetabulum of a patient.

Structures and devices of the present invention will not be limited to any particular construction materials or compositions. Materials and compositions of the invention structures can include any variety of metals, alloys, polymers, and the like, alone or in combination, that are commonly used or generally suitable for use in medical or surgical applications. Extraction devices and components thereof may be made from conventional non-absorbable, biocompatible materials including stainless steel, titanium, alloys thereof, polymers, composites and the lie and equivalents thereof.

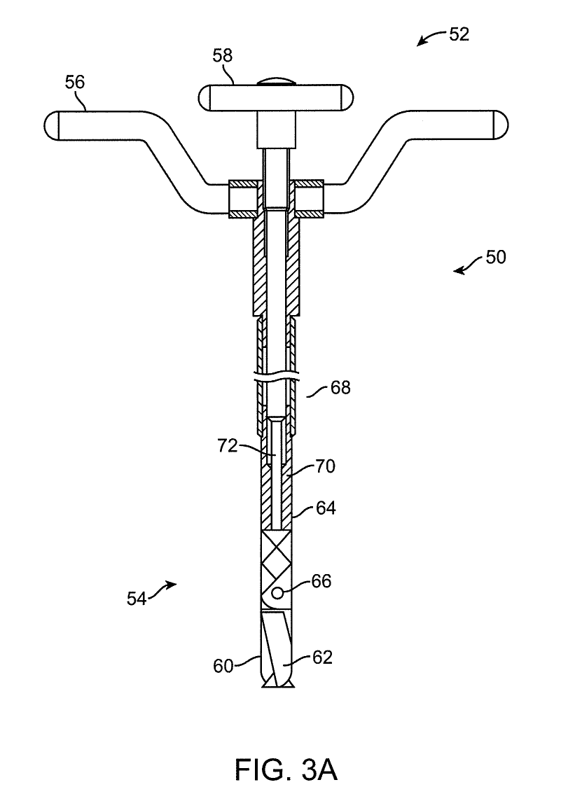

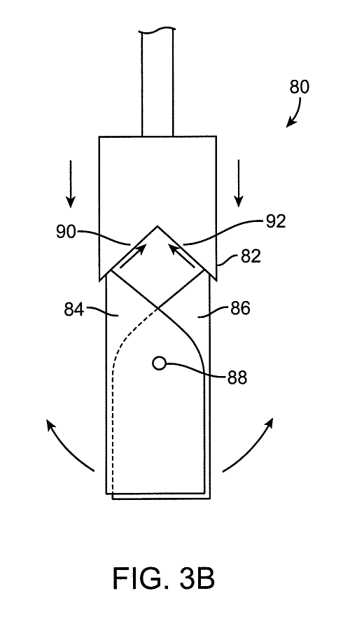

FIG. 3A shows a cross-sectional diagram of an exemplary orthopedic extraction device for engagement and removal of a bone fragment, according to an embodiment of the present invention. The device 50 includes a proximal portion 52 and a distal portion 54. The proximal portion 52 includes a handle 56 and an actuation member 58. The distal portion 54 includes deployable members 60, 62 coupled to the housing 64 about a pin 66. The device further includes an elongate body 68 having a housing 64 with an internal lumen 70. A push rod 72 is disposed in the lumen 70 of the housing 64 and configured to transmit a force or torque applied to the actuation member 58 at the proximal portion 52 along to the deployable members 60, 62 so as to elicit movement or deployment. The push rod 72 can include a single piece construction or multi-piece assembly. Push rod 72 includes a distal portion that is brought into contact with deployment members 60, 62 for actuation.

FIG. 3B is a simplified cross-sectional diagram of a deployment assembly of an extraction device, according to one exemplary embodiment of the present invention. The assembly 80 includes member 82, configured to be brought into contact with deployable members 84, 86. Member 82 may provide a distal portion of a pusher rod or component of a pusher rod assembly. Deployable members 84, 86 are positioned substantially adjacent to each other, or side-by-side, and coupled about pin 88 and configured such that each member 84, 86 is rotatably movable about pin 88. In use, application of a selected force to member 82 causes its movement toward deployable members 84, 86. Surface 90, 92 of member 82 are brought into further contact with deployable members 84, 86, respectively. Fixed pin 88 holds members 84, 86 while allowing rotation of members 84, 86 about the pin 88 as the contact surfaces of proximal portions of members 84, 86 slides along surfaces 90, 92 of member 82. The rotational movement of members 84, 86 about the pin 88 elicits movement of distal portions of members 84, 86 substantially outward or radially, so as to provide deployment of the members 84, 86.

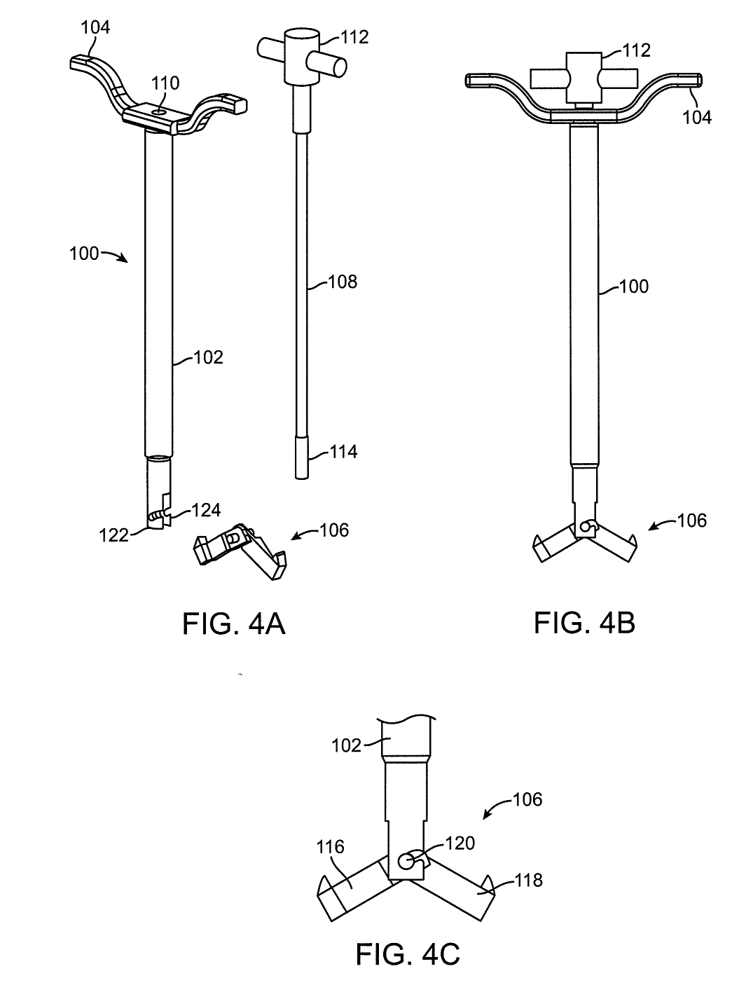

FIG. 4A shows an exploded view of a removal device, according to an exemplary embodiment of the present invention. The device includes a first unit or assembly 100 including a housing 102 having an elongate body, a proximal portion of the housing coupled to a handle 104. The device further includes a second unit or assembly forming a deployable or expandable member assembly 106, and a third unit or assembly including a actuation member assembly 108. The housing 102 of the first assembly includes a lumen 110 extending substantially between the proximal portion and the distal portion of the first unit 100. The distal portion of the assembly 100 is shown configured for removable coupling with the deployable member assembly 106. The actuation member assembly 108 includes a proximal portion with a push or torque handle 112 coupled with a proximal end of an elongate push rod assembly 114. The push rod assembly is sized or configured for disposal within the lumen 110 of the housing 102. Where the push rod is disposed 114 in the housing lumen 110 in assembly 100, the distal portion of the rod 114 is brought into direct or indirect contact with the deployable member assembly 106 operably coupled to the distal portion of the housing 102.

FIG. 4B shows an assembled device as in FIG. 4A. Deployment member assembly 106 is illustrated in a substantially deployed configuration. In use, a user can grip the handle 104 for manipulation or positioning of the device. Application of force or torque to the actuation member 112 of the proximal portion, e.g., by turning or application of a distally directed force to the actuation member 100, transfers force to the deployable member assembly for eliciting movement of members 106. For example, a user may push down or distally on the actuation member 112 to force the distal portion of the push rod 114, disposed within the lumen 110 of the elongate member 102, into further contact (directly or indirectly) with one or more surfaces of members 106. In use, deployment of members 106 brings the members into further contact with the tissue in which the distal portion of the device is disposed and allows more secure engagement between the distal portion of the device and the tissue or bone structure. With engagement of the device, bone fragment manipulation and extraction/removal can be accomplished, e.g., as described above.

The device can optionally include a structure or design such that a locking or latching mechanism such that distal advancement of the actuation assembly (e.g., advancement of the push rod distally through the lumen) triggers engagement between components of the device such that the actuation assembly is substantially prevented from recoiling or retracting back proximal toward the user. Such a locking or latching mechanism may be advantageous so as to allow continued engagement between the distal portion of the push rod and the deployable members and prevent movement of the members back to a non-deployed state. Once the actuation member is locked into position, the user can discontinue application of force to the distal portion of the actuation member while maintaining the members is a substantially deployed configuration. The locking or latching mechanism can include a releasable or reversible mechanism, where the user can disengage the locked arrangement, e.g., so as to allow the push rod to retract proximally through the lumen and reduce or discontinue force application between the distal portion of the push rod and the deployable member assembly. Disengagement of the latching mechanism, in one design example, may be accomplished by twisting or turning the actuation assembly, e.g., via application of torque to the proximal handle of the actuation assembly.

A device of the present invention can include a deployable member assembly that can reversibly be moved between a deployed or expanded configuration and non-deployed configuration. Alternatively, a device may be configured with a substantially non-reversible deployment design. For example, a deployable member assembly can include a configuration where once expansion or deployment of the members is accomplished, the members are locked into position and held substantially in a deployed configuration. In such an embodiment, the deployment assembly may be more optimally suitable for one-time use, and subsequent removal and exchange with a replacement deployable member assembly for further use of the device.

As described above, the distal portion of an extraction device of the invention will be configured for outward movement or expansion, or deployment, and engagement between expandable or deployable members and the component/tissue (e.g., bone fragment) in which the device is disposed for removal. For example, as seen in FIG. 4C, the device includes deployable members 116, 118 of assembly 106 configured for deployable movement and engagement with a bone fragment. Deployment members 116, 118 can include a proximal portion having a pin hole or via and a distal portion configured for engagement with a bone fragment, e.g., upon deployment in a tissue. In one embodiment, e.g., as illustrated in FIG. 4C, distal portions of members 116, 118 can include a hooked or sharpened portion to enhance engagement with a bone fragment.

In assembly, members 116, 118 are coupled about a pin 120 so as to form a pin hinge and allow the members to slidably move past each other between a non-deployed configuration and a substantially deployed configuration. The pin extends through via disposed in each of members and the assembly couples to the distal portion of the housing unit about the pin. In the illustrated embodiment shown in FIGS. 4A-4C, the housing includes a bifurcated distal portion including a first 122 and second 124 bifurcations with a space disposed there between. Each bifurcation similarly includes a sort of hooked portion or a slot configured for receiving the pin 120 for hinged coupling. In assembly, the members 116, 118 are coupled about the pin 120 and the distal portions of members disposed in the space between bifurcations 122, 124, with opposing end portions of the pin extending from the members positioned in a corresponding pin receiving slot.

Thus, the member 116, 118 and pin 120 assembly can be coupled to the housing in a removable fashion. For removal, the pin can be moved out of the bifurcation slots for detachment of the members 116, 118 from the housing 102. Such removable coupling may be advantageous in certain uses, for example, where members 116, 118 are deployed so as to engage the bone in which the distal portion of the device is positioned and where removal/discontinuation of the deployment force to the members 116, 118 may not sufficiently disengage the members from the bone tissue to allow removal of the device from the bone fragment. With a removable coupling configuration of the deployment assembly, the device can be manipulated such that the pin slides out of the bifurcation slots, thereby allowing the distal portion of the device to more easily be removed or disengaged from the bone fragment with members 116, 118 left in the bone. Deployment members can be disposed with the removed bone fragment or optionally separated for salvage and re-use. Thus, the extraction device may be configured for limited or single use of one or more components of the device and/or repeated use of one or more components. For example, the device may be configured for limited or one-time use of the distal portion or deployment member assembly, where the deployment assembly is detachable, e.g., in a manner described above, and replacement components (e.g., replacement deployment member assemblies) can be made available for subsequent use in conjunction with other device components.

As indicated above, a distal portion of an extraction device of the present invention is advanced into a bone fragment for removal, and advancement can include first forming an entry via or passage in the bone fragment. A drilling step can include drilling the bore along an axis which extends into the bone fragment at a selected entry point. In femoral head extraction applications, a bore can be formed along an axis extending into and through at least a portion of the femoral head. Generally, drilling will be selected such that the bore does not pass through the femoral head and into the acetabulum. Various drilling entry points or locations may be selected, and can include drilling into the femoral neck as well as drilling an entry point into the femoral head but displaced from the femoral neck.

A conventional orthopedic drill or boring device can be utilized, as well as a conventional drill bit selected to produce the desired hole or passageway in the bone fragment. In certain embodiments, the present invention may include a drilling control means for controlling positioning of the drill and/or controlling or restricting depth penetration of drilling such that the passageway does not penetrate substantially beyond the desired depth. A control means can include, for example, use of a calibrated drill with a depth penetration restrictor. In a femoral head drilling application, depth penetration restriction may be desired and selected so as to prevent the distal end of the drill bit from advancing beyond the desired depth and, for example, passing through the femoral head and into contact with the acetabular side. Drilling control can further include use of a suitable drill guide or control member.

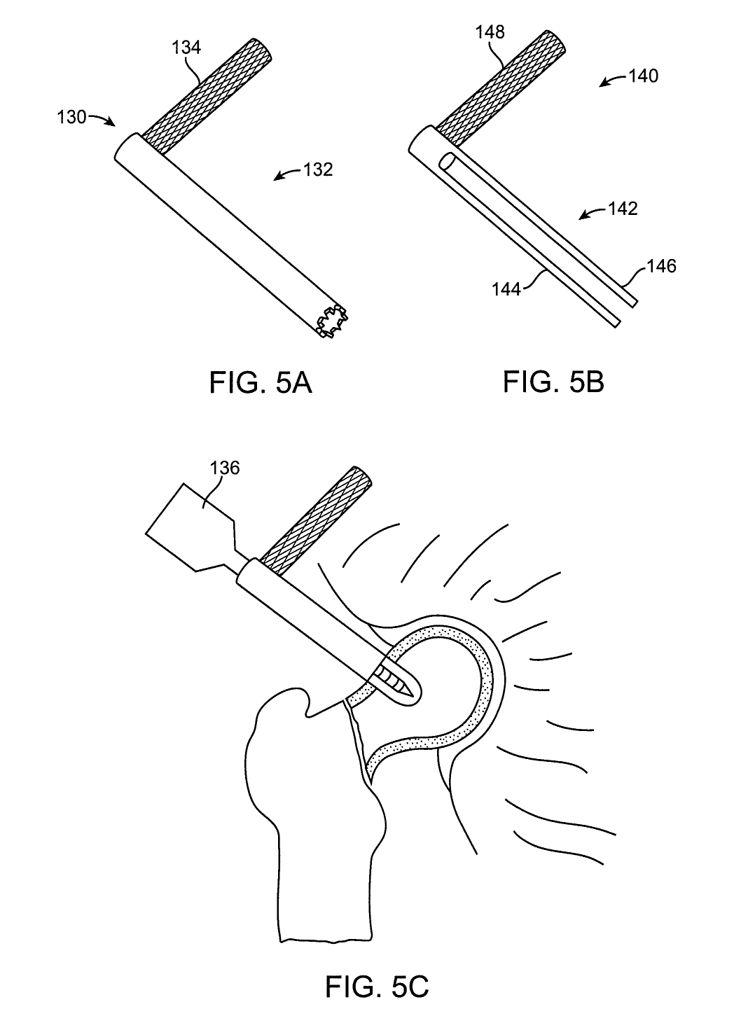

FIGS. 5A and 5B illustrate exemplary drilling control apparatus or drill guides/members for directing and/or controlling drilling into a bone fragment. FIG. 5A shows a guide 130 including an elongate tubular member 132 having a handle 134 coupled to a proximal portion of the member 132. The distal end of the tubular member includes serrated or corrugated surface for improved engagement between the distal end and a bone surface to decrease sliding of the distal end when positioned on a bone surface. In use, the distal end of the guide is positioned at a desired location on a bone surface (see, e.g., FIG. 5C), with positioning or stabilization of the guide including gripping of the handle by the user. A bit of a drilling device 136 is inserted in the proximal end of the guide and advanced through the tubular member and into drilling contact with the bone fragment. The length and/or sizing of the guide can be selected and/or coupled with a drill bit (e.g., selected drill bit sizing/configuration) to control depth penetration of the drill bit, as contact between the proximal end of the guide and the drilling apparatus will prevent further advancement of the drill bit distally.

A drill guide elongate member can include a tubular member with a substantially continuous housing or a discontinuous housing. For example, one or more windows or openings along a housing length may be selected in a guide design, and may be desired, for example, for improved user viewing of a drill bit positioning within the guide. FIG. 5B illustrates an exemplary drill guide 140 having an opening to the housing lumen. The elongate portion 142 of the guide 140 includes opposing prongs 144, 146 coupled proximally to a handled portion 148. The distal portion is configured for positioning on a bone surface as described above, and can include distal end contact surface of a variety of configurations. Use of the guide in drilling is similar to as described above (see, e.g., FIG. 5C).

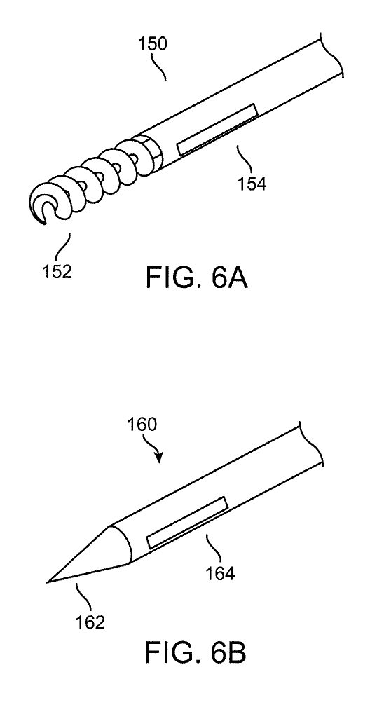

Devices and structures of the present invention can further include a distal portion configured to further facilitate entry and/or advancement of the device through a tissue and into a bone fragment for extraction as described above. Use of a sharpened or threaded distal tip or portion of an extraction device, for example, may allow the distal portion of a device to advance more easily along a passageway or drill hole, or in the absence of a pre-formed passageway, and into a bone tissue. FIG. 6A illustrates a distal portion of an extraction device 150 having a threaded or screw portion 152. In use, advancing of the device can include applying a rotational movement to the device so as to embed or screw the distal portion further into the target bone fragment. Members (not shown) are then deployed or expanded outward from openings 154 in the device housing for further engagement of the bone fragment and removal as described. FIG. 6B shows a distal portion of an extraction device having a sharpened or pointed distal end 162, and having openings 164 in the housing for deployment of members and further engagement of the bone fragment similar to as described above.

The structures and methods described herein are particularly well suited for removal of a femoral bone fragment or femoral head from the acetabulum of a patient, e.g., in a hip replacement procedure. Although the extraction devices and methods of the present invention are described primarily in the context of fractures of the proximal femur and bone removal from the acetabulofemoral joint, the methods and structures disclosed herein are intended for application in a wide variety of bones and bone removal applications, as will be apparent to those of skill in the art in view of the disclosure herein. In the broadest sense, methods and structures can be designed and/or utilized for separating two components from one another. In one example, the present invention can include separating a foreign material or implant from a tissue, such as removing a cement or implant from a bone or bone canal. As such, the term bone fragment as used herein may in some instances refer to an implant or foreign material (e.g., bone cement, etc.) disposed in a patient's body and amenable to removal according to the methods described herein.

Methods of the present invention can include use of a single extraction device for removal of a bone fragment or can include use of a plurality of devices for a removal. In one example, a first extraction device can be inserted in a femoral head in a first location for engagement with the bone. A second extraction device can then be inserted and advanced into the femoral head at a different location for engagement with the femoral head. With the plurality of devices engaged with the bone fragment, the femoral head can be removed similar to as described above.

In the application of femoral head removal from the acetabulofemoral joint of a patient, as noted above, methods herein can include removal of a head of a femur that has been subject to fracture or osteotomy. Methods of the present invention will also include removal or dislocation of the femoral head from the socket absent fracture or osteotomy, or prior to osteotomy.

One or more structures as described herein may be provided in the form of a kit. A kit may be assembled for portability, facilitating use in a surgical setting, and the like. This kit typically includes an extraction device of the present invention, and the extraction device may be provided in a fully assembled, partially assembled, or non-assembled configuration. As indicated, a device of the present invention may be configured or of a design that one or more components of the extraction device have a limited or single use, or are replaceable. As such, a kit can include an extraction device with one or more replacement components, such as one or more replacement deployable member assemblies. In one example, a kit may be assembled for a double hip replacement surgery such that a first portion of the device can be utilized for removal of both femoral heads of the patient and a second component (e.g., deployable member assembly) that is removably couplable, with the kit including a replacement second component. A kit may include pre-sterilized components or device(s), as well as sterilized packaging.

The components of the present invention may be sterilized (and will generally be sterilizable) by any of the well known sterilization techniques, depending on the type of material. Suitable sterilization techniques include heat sterilization, radiation sterilization, chemical/gas sterilization, and the like.

The specific dimensions of any of the extraction devices, systems, and components thereof, of the present invention can be readily varied depending upon the intended application, as will be apparent to those of skill in the art in view of the disclosure herein. Moreover, it is understood that the examples and embodiments described herein are for illustrative purposes only and that various modifications or changes in light thereof may be suggested to persons skilled in the art and are included within the spirit and purview of this application and scope of the appended claims. Numerous different combinations of embodiments described herein are possible, and such combinations are considered part of the present invention. In addition, all features discussed in connection with any one embodiment herein can be readily adapted for use in other embodiments herein. The use of different terms or reference numerals for similar features in different embodiments does not necessarily imply differences other than those which may be expressly set forth. Accordingly, the present invention is intended to be described solely by reference to the appended claims, and not limited to the preferred embodiments disclosed herein.

What is claimed is:

1. A method of removing a bone fragment from a body of a patent, comprising:
advancing a distal portion of an orthopedic extraction device into a bone fragment disposed in a bone mass in the body of the patient, the extraction device comprising an elongate body, a proximal portion and the distal portion comprising a deployable member assembly;
actuating the deployable member assembly of the device with the distal portion of the device positioned in the bone fragment so as to engage the bone fragment with the device; and
applying a force or torque to the device engaged with the bone fragment so as to remove the bone fragment from the bone mass of the patient.

2. The method of claim 1, wherein the bone fragment comprises a femoral head disposed in an acetabulum.

3. The method of claim 1, advancing comprises drilling or boring a passageway in the bone fragment and then advancing the distal portion of the device into the passageway.

4. The method of claim 3, wherein the drilling or boring comprises positioning a guide member on the bone fragment so as to guide a drilling or boring device into contact with the bone fragment.

5. The method of claim 1, wherein the distal portion of the extraction device comprises a threaded portion.

6. The method of claim 5, wherein advancing comprises rotating the distal portion so as to screw the distal portion into the bone fragment.

7. The method of claim 1, wherein actuating the deployable member assembly comprises eliciting movement of the assembly between a non-deployed configuration and a deployed configuration.

8. The method of claim 1, wherein the proximal portion comprises an actuation member coupled to the deployable member assembly and actuating the assembly comprises applying a selected force or torque to an actuation member at the proximal portion so as to elicit movement of the assembly between a non-deployed configuration and deployed configuration.

9. The method of claim 1, further comprising detaching one or more components of the device for replacement following use of the device.

10. An orthopedic extraction device for removing a bone fragment from a bone mass, the device comprising:
an elongate body coupling a proximal portion and a distal portion of the device, the distal portion configured for advancement into a bone fragment disposed in a bone mass of a patient, the distal portion comprising deployable member assembly, and the proximal portion comprising an actuation member coupled to the deployable member assembly such that application of a selected force or torque to the actuation member elicits movement of the assembly between a non-deployed configuration and a deployed configuration for engagement of the device with the bone fragment.

11. The device of claim 10, further comprising a proximal handle configured for user manipulation or position control of the device.

12. The device of claim 10, wherein deployable members each comprise a hooked distal portion and are coupled proximally about a pin hinge.

13. The device of claim 10, the device comprising a push rod disposed in a lumen of an elongate body housing.

14. The device of claim 13, wherein the actuation member is coupled to the push rod such that application of the selected force or torque to the actuation member brings a distal portion of the push rod into further contact with the deployable member assembly so as to elicit movement of the assembly.

15. The device of claim 10, further comprising a threaded or sharpened portion disposed distally to the deployable member assembly.

16. The device of claim 10, wherein the deployable member assembly is removably coupled to the elongate body.

17. The device of claim 10, the device comprising one or more single or limited use components.

18. A system for removing a bone fragment from a bone mass, the system comprising:
an orthopedic extraction device comprising an elongate body coupling a proximal portion and a distal portion of the device, the distal portion configured for advancement into a bone fragment disposed in a bone mass of a patient, the distal portion comprising deployable member assembly, and the proximal portion comprising an actuation member coupled to the deployable member assembly such that application of a selected force or torque to the actuation member elicits movement of the assembly between a non-deployed configuration and a deployed configuration for engagement of the device with the bone fragment; and
a guide member for directing a drilling or boring device into contact with the bone fragment.

19. The system of claim 18, wherein the deployable member assembly is removable and the system further comprising one or more replacement deployable member components.

20. The system of claim 18, the guide member comprising an elongate housing having a proximal portion coupled to a handle and a distal portion configured for engagement with a surface of a bone fragment.

21. The system of claim 20, wherein the housing is a continuous tubular housing or a discontinuous housing comprising substantially opposing prongs.
